(19) United States
(12) Patent Application Publication
(10) Pub. No.: US 2016/0324719 A1
(43) Pub. Date: Nov. 10, 2016

(54) PORTABLE COMPRESS DEVICE AND METHOD OF USE

(71) Applicant: Ekaabo Inc., Richmond Hill (CA)

(72) Inventors: Abosede Titilola Badmus, Richmond Hil (CA); Derron Mahoney Lyn, Richmond Hill (CA); Chris Pearen, King City (CA); Robert Dickie, King City (CA); Gerard Campeau, Aurora (CA)

(21) Appl. No.: 15/216,283

(22) Filed: Jul. 21, 2016

Related U.S. Application Data
(63) Continuation-in-part of application No. PCT/CA2015/050003, filed on Jan. 5, 2015.

(51) Int. Cl. A61H 23/00; A61F 7/02; A61F 7/00

(52) U.S. Cl. CPC A61H 23/00; A61F 7/007; A61F 7/02; A61F 2007/0052; A61F 2007/0087; A61F 2007/0078; A61F 2007/0084; A61F 2007/0021; A61F 2007/0075; A61F 2007/0228; A61H 2201/02

(57) ABSTRACT

A portable compress device includes a main body, a power source, an electric means for generating a temperature gradient on a plurality of surfaces, a controller means coupled with the electric means and the power source, a thermal sensor device configured to be in proximity to a user's skin at a treatment area, and a plurality of pad attachment and detachment means for attaching and detaching a pad assembly to and from the plurality of surfaces, wherein the pad assembly is for moistened or dry application on the treatment area.

### FIELD OF TECHNOLOGY

The present disclosure relates to skin care for humans and animals and, more particularly, to a portable compress device.

### BACKGROUND

Efforts to apply sustained thermal compress therapy are known, such as a heated or cooled gel pack, or by exothermic reaction from chemical compounds, or electric powered warmers. Such methods often require an external source (e.g. a microwave oven or refrigerator) to obtain the desired thermal gradient for multiple usage. In addition, most known uses for thermal compress therapy provide dry compressing for the relief of muscle aches and pain over relatively large surfaces of the body.

Other efforts, such as U.S. Pat. No. 4,915,108 entitled ‘Hot and cold compress device,’ for example, do not contemplate use of moisture while compressing, among other limitations.

The treatment of skin ailments with antibiotics alone may be inadequate. While the home-based methods of performing hot soaks or use of a gel packs may be suitable when resources are available, a clear need exists for an alternative or a more convenient method to deliver a thermal compress that is portable and safe.

Improvements in portable compress devices are desirable.

### BRIEF DESCRIPTION OF THE DRAWINGS

Examples are illustrated with reference to the attached drawings. It is intended that the examples and figures disclosed herein be considered illustrative rather than restrictive.

FIG. 1 is a front perspective view of an exemplary portable compress device;

FIG. 2 is a rear perspective view of the portable compress device of FIG. 1, showing an optional dispenser to allow the delivery of fluids to a pad in contact with the skin;

FIG. 3 is an exploded view of the portable compress device of FIG. 1;

FIG. 4 is a front view of the portable compress device of FIG. 1;

FIG. 5 is a sectional view according to the cross-section line in FIG. 4, showing the components of the portable compress device of FIG. 1;

FIG. 6 is a perspective view of the portable compress device of FIG. 1, showing the operation of a sleeve door to access a pad;

FIG. 7 is a perspective view of the portable compress device of FIG. 1, showing how a pad is placed;

FIG. 8 is a perspective view of the portable compress device of FIG. 1, showing the operation of a sleeve door to secure a pad;

FIG. 9 is a bottom perspective view of the portable compress device of FIG. 1;

FIG. 10 is a perspective view of a portable compress device according to an alternative example, with three surfaces;

FIG. 11 is a perspective view of a portable compress device according to a further alternative example, with dual surfaces; and

FIG. 12 is a perspective view of a portable compress device according to a further alternative example, with adjustable prongs.

### DETAILED DESCRIPTION

The present disclosure relates to a portable compress device in which a region of skin is subjected to thermal energy and/or moisture to treat skin.

A portable compress device includes a main body, a power source, an electric means for generating a temperature gradient on a plurality of surfaces, a controller means coupled with the electric means and the power source, a thermal sensor device configured to be in proximity to a user's skin at a treatment area, and a plurality of pad attachment and detachment means for attaching and detaching a pad assembly to and from the plurality of surfaces, wherein the pad assembly is for moistened or dry application on the treatment area.

One example of use is for the treatment of simple boils (i.e. small boils which do not require professional medical attention). The use of the term ‘boil’ extends to furuncle, carbuncles, cysts, blepharitis, chalazion, and styes. The basic principle is to safely heat up the surface of the skin over a period of time to increase blood circulation, which accelerates the formation of a ‘head’ (i.e. pus). Boils are an infection that may be caused by the bacterium Staphylococcus aureus and typically occurs within a hair follicle of a person or animal (the use of the term ‘boil’ extends to furuncle, carbuncles, cysts, blepharitis, chalazion, and styes). This is unlike acne which occurs when a hair follicle or oil duct becomes clogged and infected. The treatment of acne includes, for example, antibiotics, retinoid, antimicrobials such as benzoyl peroxide, laser treatment, and short pulses of thermal energy from UV light radiation or heating elements. Methods used to treat acne condition are aimed at inhibiting growth of the acne bacteria, unplugging the follicle, or to decrease sebum production. The primary treatment for simple boils (i.e. small boils which do not require professional medical attention) is draining. Draining may occur when the center of the boil softens and becomes filled with infection-fighting white blood cells, bacteria, and proteins known as pus. The pus forms a “head” on the boil and may be surgically opened or spontaneously drain out through the surface of the skin. An attempt to drain or lance the boil prior to the formation of a “head” is not helpful. To hasten the boil coming to a “head”, heat may be applied to the affected area to increase blood circulation and the presence of antibodies and white blood cells to fight off the infection. The primary method for warming the tissue has been a home based treatment involving the repeated application of a clean cloth dampened in warm to hot water for 15 to 20 minutes at least four times daily. This method is known as hot moist compress or hot soaks where the added moisture allows better penetration of the heat.

A second example of use is for the treatment of breast-feeding mothers experiencing symptoms such as engorged tender breasts, attenuated milk flow, plugged milk ducts, and/or discomfort from the symptoms of infection such as mastitis. Further examples of use extend to the following symptoms and conditions: arthritis, blepharitis, C-section inflammation/scar, fever, hot flashes, insect bites and stings, menstrual cramps, migraines or headaches, repetitive strain injury, and sports injuries such as sprains, and strains. Generally speaking, the portable compress device is suitable for providing hot and cold therapies to reduce inflammation and discomfort.

According to one example, a portable compress device includes a heat pump coupled to a controller, and may be operated such that blood flow is increased quickly in a section of skin by raising the surface temperature of the skin. The tissue of the skin subjected to localized heating for a given time elevates the temperature of the skin in that location. This elevation of skin temperature corresponds to increased blood flow to the location by making the capillaries dilate, thus leading to an increase of immune factors arrival. In the case of treating a boil, this elevation may also lead to the drawing of cellular waste, bacteria, etc. to the surface of the skin to form a ‘head’ (i.e. pus) on a boil. Furthermore, the same device may be used to provide cold treatment to the boil to help relieve pain or other treatment. It is also contemplated that operation of the portable compress device as described herein may include treating the skin with 10 to 15 minutes application of thermal energy. Other treatment durations are included within the scope of the present specification.

According to disclosed examples, the portable compress device may be hand held or strapped to the body to allow portable hot or cold compressing, and may incorporate a means of adding moisture by transferring fluid from an attached reservoir to a pad in contact with the skin. The use of the term ‘pad’ extends to a disposable pad or a reusable pad with or without a disposable sleeve, and where the area in contact with the skin is made of natural fibers, according to one example. The use of the term ‘pad assembly’ extends to a structure including a pad and other components to facilitate attaching the pad assembly to the portable compress device.

According to one example, when in contact with the skin, thermal conduction to the moist pad elevates the temperature of the skin to cause an increase in blood flow. Where the skin includes a lesion such as a boil, thermal conduction may lead to the formation of a ‘head’ on the boil, a salutary occurrence for treating the lesion.

According to one example, the portable compress device includes one or more energy sources that may be battery operated and uses a heat pump, such as a Peltier effect device, where the heat or cold generated may be controlled by varying the current direction and intensity flowing through the portable compress device. The specification is not limited to the use of Peltier effect devices; other energy sources may be used without departing from the scope of the present specification. Furthermore, various ingredients may be added to the pad in contact with the skin (e.g. Tree oil or drawing salves) and/or in the reservoir solution to, for example, speed up healing, relieve discomfort or inflammation, and the like. For example, given that the treatment of boils would benefit from a highly concentrated saline solution in which pathogenic bacteria cannot survive, such a solution may be stored in the reservoir for a period of time.

In accordance with one example, other aspects of the portable compress device may incorporate one or more controller means, such as a microprocessor, to regulate the temperature at the surface of the skin and the treatment time as indicated by user selection inputs, or based on pre-defined ranges. As a result, since the total heat transferred, or removed, will not damage skin generally speaking, typically in the range of 7 to 60 degrees Celsius, the method and device are safe for regular use as a hot or cold moist compress on external skin surfaces of humans and animals.

An aspect of the present specification further provides a portable compress device and a method for treating skin which is different from traditional hot moist compressing (i.e., repeated application of wash cloth soaked in warm or boiling hot water), and enables a portable and convenient thermal energy source to treat skin ailments such as, for example, boils. Details of the portable compress device will become apparent from the following description and accompanying drawings.

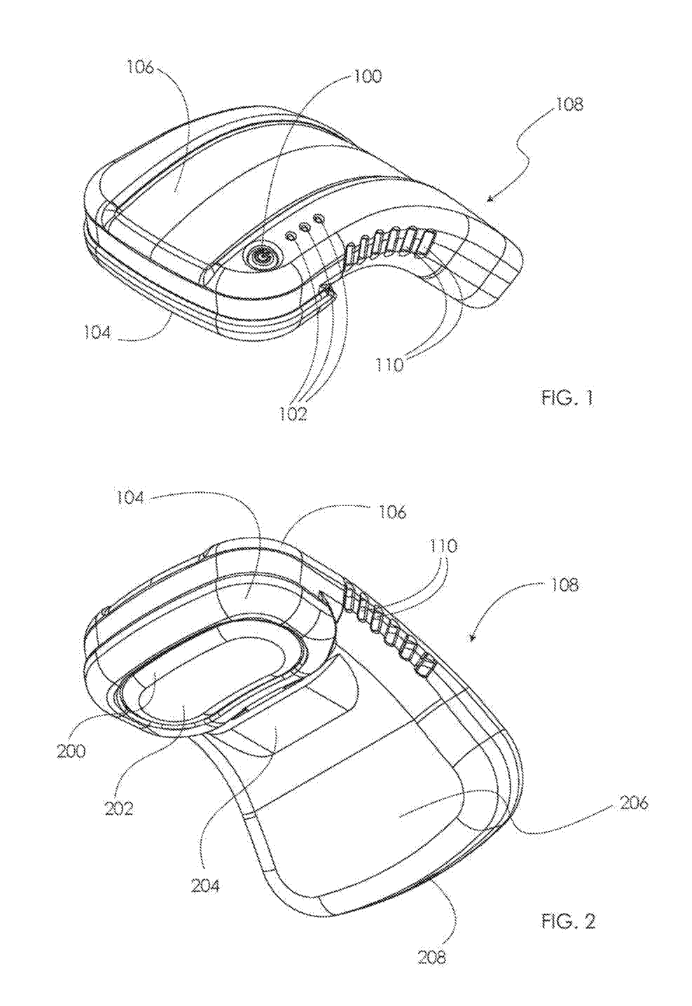

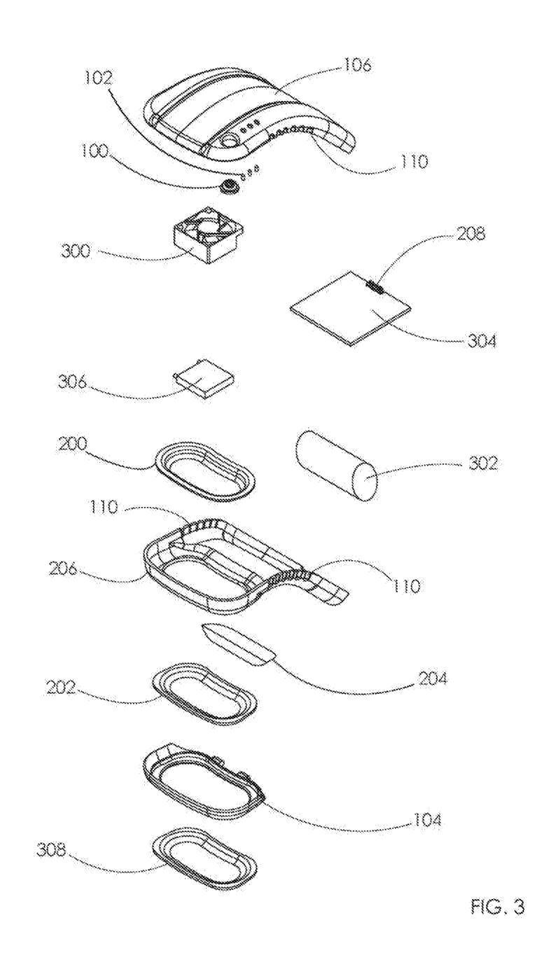

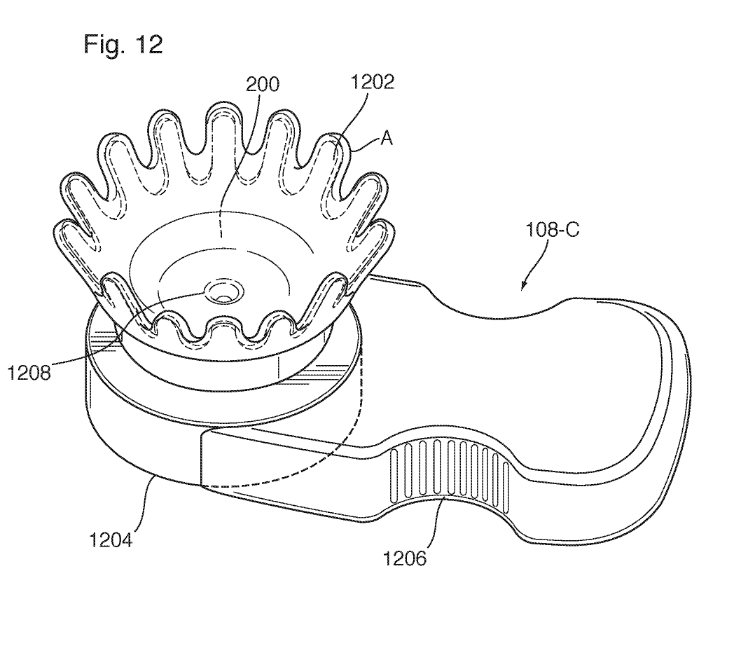

A top and bottom view of a portable moist compress device for providing localized moist heating or cooling to the skin of humans and animals is provided in FIG. 1 and FIG. 2. According to one example, the device includes a main body that may be a hand-held enclosure 108 including a power source, most typically a rechargeable or disposable battery 302, a Peltier effect device 306 adjoined to a surface 200, a controller means 304, and a liquid dispenser 204 for transferring fluid to a pad 202 (e.g. cotton). In other examples, the hand-held enclosure 108 is omitted or is detachable from the main body (as shown in FIG. 12), permitting the main body including the surface(s) and pad(s) to be positioned or attached to the treatment area (e.g. by a retention strap or adjustable prongs) for remote control (e.g. by an application on a portable electronic device such as a smartphone). Components of the portable compress device, including the main body and/or hand-held enclosure 108 may be manufactured from food grade or medical grade material.

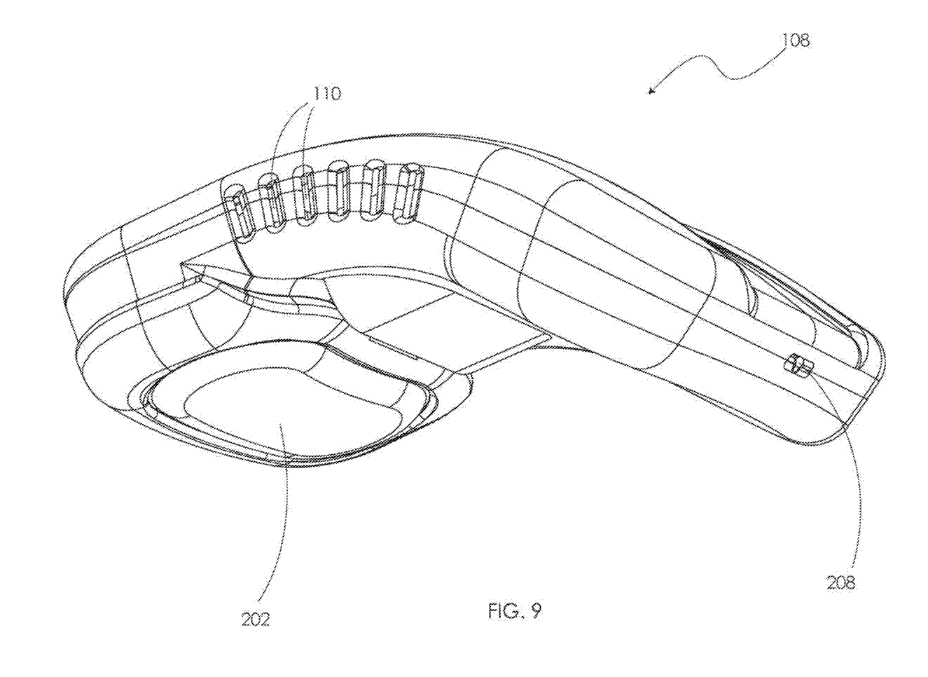

FIG. 9 shows a method of charging the battery using a micro USB port 208, and a micro USB charger (not shown). Alternatively, the power source may include a two-prong plug that is used with an 110V AC or similar outlet, and means for converting the input voltage to a form compatible with the battery type. The power source may, alternatively, include a solar or other renewable energy source (not shown), and means for converting the input voltage to a form compatible with the battery type. According to one example, a solar panel may be integral with the portable compress device or separate from the device such as on an optional docking station (not shown).

The circuit board may be activated with a power/function control means (e.g. button, dial, switch, etc.) 100 where current begins to flow through a solid-state active heat pump (e.g. a Peltier effect device as shown or thermoelectric heat pump). The flow of current through the Peltier effect device is used to alter the temperature of the heating surface. This effect is used to generate, or remove heat, at a junction when a current flows through that junction between two different conductors. The present device uses this thermoelectric effect for warm and cold applications to the skin by regulating the intensity and direction of the current. A thermal sensor device (not shown) such as a thermocouple or resistive thermal device is used in a feedback circuitry to implement a highly stable temperature profile that may maintain the temperature of the heating surface within +/−0.5 degree Celsius. The thermal sensor device may be configured to measure the temperature at the heating surface of the device, or the temperature at the surface of the skin in contact with the device. A fan 300 and vent openings 110 shown on the top body 106 and bottom body 206 may be used to dissipate heat and allow lower temperatures to be realized based on the Peltier effect. The heating surface is used to transfer the thermal energy from the heat pump to the pad secured on its surface.

In one example, the portable compress device and/or optional docking station, including the electronics components, may be water-proofed using a super-hydrophobic coating or other water-proofing technique.

FIG. 1 shows an example of the power/function button 100 located on the exterior surface of the device whereby the user may manually power on/off the device, and select a thermal gradient profile from a number of available thermal profiles. Each thermal profile may include characteristics such as treatment duration, temperature, and hot/cold cycles. An indication may also be used to convey relevant information such as power, when selected temperatures are reached, battery life, number of applications used, alarms, and end of treatment session. For example, visual indication is provided by the LED sequence 102, and may also include audible and vibratory indications. In an alternate example, user input may be made using an analog or digital input device, and LCD screen to provide the indications.

In a further alternate example, a portable electronic device (e.g. a Bluetooth mobile phone, wearable or other electronic device) executing a software application or ‘app’ may interface with the portable compress device to provide wireless control and display of the portable compress device, or vice versa.

Peak temperatures for the portable compress device may be selected to reduce or attenuate the risk of skin damage. For example, the selection of peak temperatures may be based on a study on temperature limits for skin contact with hot and cold objects published by Eugene Ungar and Kenneth Stroud for NASA/Johnson Space Center (A New Approach to Defining Human Touch

Temperature Standards). Other studies investigating skin damage due to temperature resulted in similar findings. From these studies, it was determined that temperature limits of 45° C. (113° F.) to 10° C. (50° F.) may be used for commonly used material indefinitely while avoiding damage to the skin. Higher temperature values were also attainable for shorter time periods. For lower temperatures, 7° C. and below may result in skin numbness and 0° C. and below resulting in frostbite. For the portable compress device, in order to safely apply a higher temperature of 60° C., input from the temperature sensor may be used to regulate the cycle of the current flow in the heat pump to provide the appropriate temperature versus time profile to prevent skin damage. For cold applications, 7° C. is used, and in this case a minimum time is enforced between cold applications. It has been discovered that human skin may be able to tolerate temperature limits beyond the range specified by Ungar et al. depending on the characteristics of the subject under treatment and the duration of use. As well, it is noted that the sensed temperature may be different from the temperature of the surface. For clarity, use of the term peak temperatures is made with reference to the range of temperatures that enable safe contact between the pad (or pad assembly) and the skin of the subject under treatment for the desired duration of treatment. The present specification is not intended to be limited to the ranges defined above.

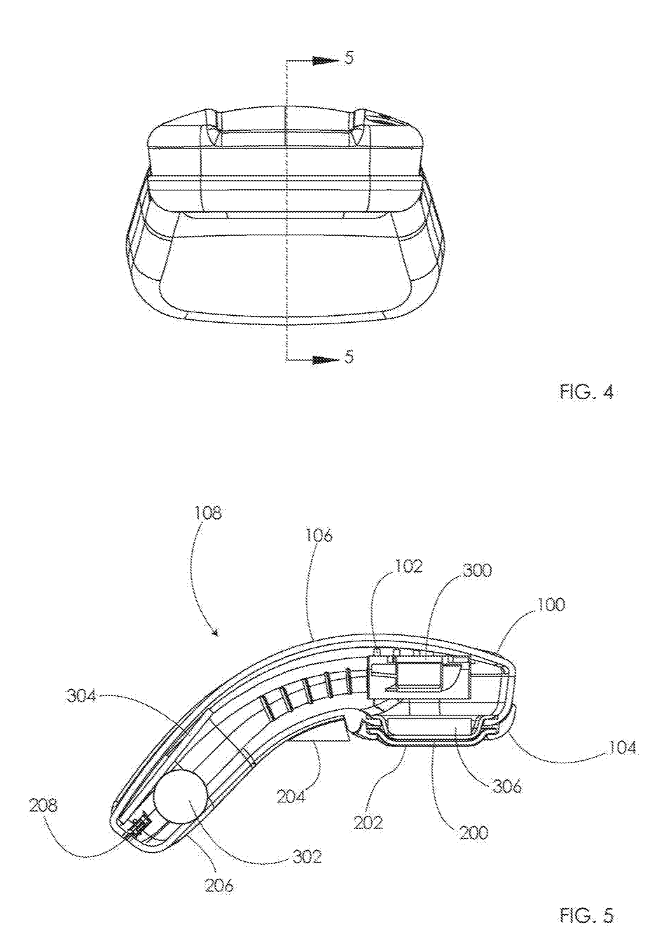

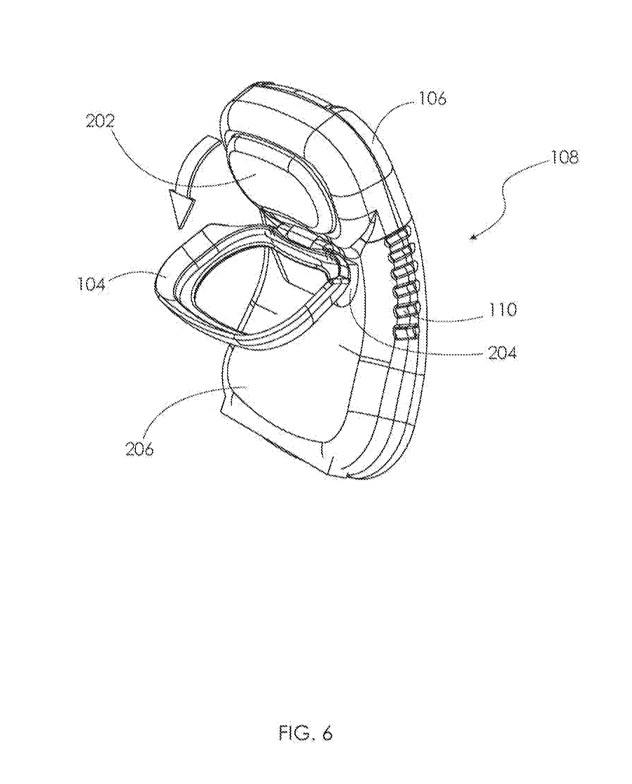

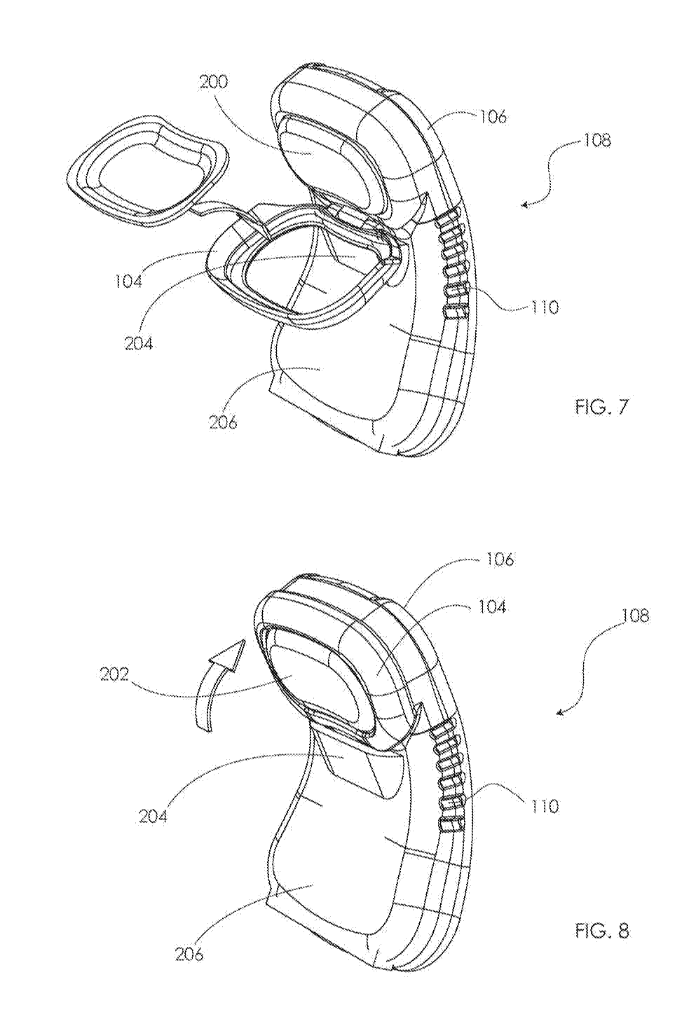

FIG. 4 to FIG. 5 show a cross sectional view of the portable compress device where the top body 106 and bottom body 206 isolates the electrical components from any moisture produced by the heat pump, the pad, or from any body fluids during use. As shown in FIG. 1, the shape of the portable compress device, according to one example, is generally a curved configuration with a furrow along the length of the top body for easier handling around body parts, and for hand placement during use. The area used for holding the device, including the furrow surface along the length of the top body, is preferably provided with a non-slip surface. Additionally, the area in the vicinity of the pad is waterproofed for cleaning. FIG. 6 to FIG. 8 show an exemplary mechanism to secure and remove the pad using a sleeve door on a hinge 104. A retention strap (not shown) may be configured along the length of the device enabling the user to obtain more or less compression of the device on various parts of the body during use. A cover 308 may be used to protect the pad when not in use.

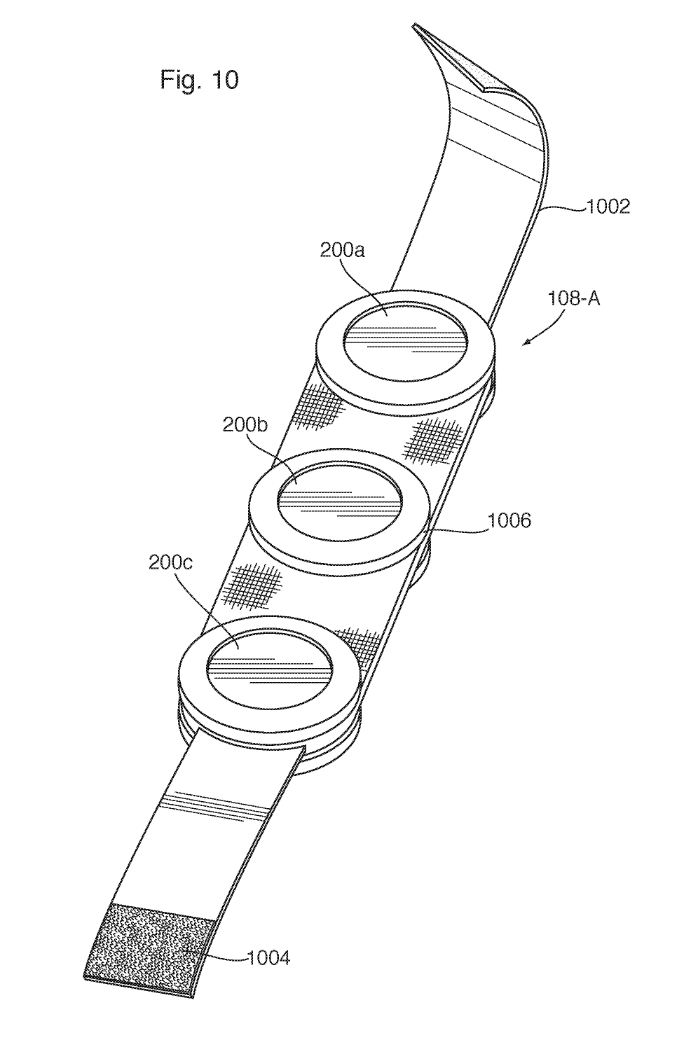

Turning now to FIG. 10, in one example, the portable compress device includes three surfaces 200a, 200b, 200c on the main body 1006. Additional surfaces may be arranged on the opposing side of the main body as shown in FIG. 10. The surfaces are arranged for application on a wide, or greater, treatment area. Fewer or great surfaces may be included without departing from the scope of the present specification. The plurality of surfaces may be arranged in a row, a column, a triangle configuration, or some other configuration. The surfaces may be of uniform size or vary in size relative to one another. The spacing between the three surfaces may be varied in width or adjusted. A retention strap 1002, which may be flexible or elastic, can provide a fit such that the surfaces and associated pads are in proximity to the treatment area.

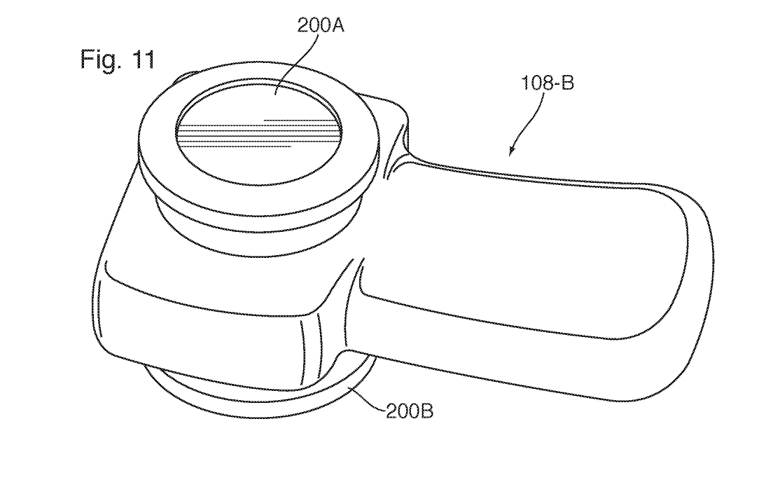

Now with reference to FIG. 11, in a further example, the portable compress device may include two surfaces 200a and 200b and two pad attachment and detachment means corresponding to the two surfaces. The surfaces may be arranged on opposing sides of the main body as shown in the figure. The first surface 200a may be for hot therapy and the second surface 200b may be for cold therapy. A pressure sensor (not shown) may detect which side is being used. In operation, current from the energy source may be directed to the surface being used. This configuration leverages the Peltier effect device which produces hot and cold at opposite sides.

FIG. 12 shows a further example of the portable compress device including a main body consisting of a hand-held enclosure 1206 and a detachable portion 1204. According to this example, the pad attachment and detachment means includes an inverted dome 1208 and a plurality of adjustable prongs 1202 for treating a treatment area that may include protruding skin such as a breast. The pad assembly (shown as “A” on FIG. 12) may correspondingly include a plurality of openings to accommodate the prongs. The prongs 1202 may be adjustable (not shown) by bending for example. The arrangement of the prongs 1202 may help treat areas of the body that bend such as knees, elbows, and the like, or that generally can be placed in whole or in part within the inverted dome shape or other opening of the main body of the portable compress device.

In some examples, the curvature along the length of the device may be made adjustable to allow for better placement on the user's skin. In other examples, the components consisting of the

Peltier effect device, fan, heating surface, temperature sensor, and pad may be physically separate from the components related to power and control (e.g. controller circuit board, battery, and program selection means). In this example, the separated components will remain operationally coupled by means such as wires, or interface with each other wirelessly (e.g. Bluetooth) in order to provide remote operation

Numerous variations of the mechanism to secure and remove the pad are possible to further promote hygienic handling. For example, the sleeve door 104 may be replaced with one that is snapped shut to secure the pad in place. Other means for securing and removing the pad may include the pad backing and heating surface 200 consisting of a fabric hook and loop fastener method. In another variation to secure and remove the pad, it may be permanently or detachably connected to the device by a suitable structure formed on the pad backing. This structure is then interfaced to by the device to allow mechanical attachment and detachment of the pad without the user touching the pad's surface.

Furthermore, the structure formed on the pad backing used to provide a means of attachment and detachment to/from the device may also be used as a means of stacking the pads one on top of the other to allow access to the structure formed on the pad immediately below the pad being removed.

The portable compress device, according to one example, may also incorporate thermal conductive material interfacing the heating surface and the pad to provide additional comfort to the user by conforming to the shape of the applied area on the user's skin (e.g. a TIM-GEL PAD which is a soft thermally conductive silicone material with thermal conductivity of 3 W/m°K).

In one example, the pad material may be made from natural fibers, and impregnated with a drawing salve (e.g. Ichthammol drawing salve) to help bring the boil to a head. The outer ridge of the pad's circumference may have an adhesive to keep the pad and drawing salve in contact with the skin. According to one example, the natural fibers may be a plant-based polyurethane foam.

In addition to temperature change, another aspect of the user's treatment is moisture to allow better penetration of the heat or cold. Given that some conditions treated by the device may benefit from a highly concentrated saline solution in which pathogenic bacteria cannot survive, a refillable reservoir or liquid dispenser 204 to store such a solution may be provided. While such a dispenser may be used to pre-moisten the pads prior to insertion in the device, an alternative method is shown in FIG. 5 where the solution from the liquid dispenser, secured adjacent to the heating surface 200, may be applied to the pad by user input (e.g. when a button on the dispenser is pressed).

In use according to one example, the portable compress device will quickly heat or cool to predefined set points (e.g. high, medium, low, or cold), or to a user-specified temperature within the range for safe skin application as previously defined. Indication is provided when the set temperature is obtained. The time period for applications may also be predefined, or user selected. In the case of consecutive cold applications, a minimum time between applications is enforced. The user adds liquid solution from the dispenser to the pad and places the padded surface over the treatment area for the time duration as defined by the selected program. The device may then be automatically powered off, and the pad detached from the device.

For the treatment of boils, for example, various ointments and drawing salve solutions may be impregnated in the pad to bring the lesion to a head and speed healing. The pads once detached from the portable compress device after the warm compressing treatment has ended, may remain on the treated area through a means of adhering the pad to the skin.

For the treatment of a breast-feeding mother's skin, for example, the pads may be individually packaged using natural, gel-free organic cotton shell that is infused with organic, natural nipple cream containing ingredients safe for the mother and the baby. The pads may be pre-moistened in a sterile saline solution to assist healing of damaged nipples, or dry. The pads may be hygienic sterile and disposable pads.

According to disclosed examples, the present specification provides a portable compress device including a main body, a power source; at least one electric means for generating a temperature gradient on a plurality of surfaces, a controller means coupled with the electric means and the power source, a thermal sensor device configured to be in proximity to a user's skin at a treatment area, and a number of pad attachment and detachment means for attaching and detaching a pad assembly to and from the surfaces. The pad assembly may be moistened for application on the treatment area.

In one example, the device may include a number of heat sinks and/or fans for regulating the temperature of the main body, which may be hand-held. The main body may include a rubberized handle. The main body and the pad assembly may be curved to facilitate ergonomic operation. The device may include a vibration motor to provide relief from discomfort or stimulation. The pad assembly may be a permanent gel pack that is integral. The pad assembly may be a disposable gel pack.

In some examples, the power source may be selected from one of disposable batteries, rechargeable batteries, AC power, solar power, USB power, and the like.

According to one example, a portable compress system is disclosed that includes the portable compress device and a docking station. The power source of the device may include rechargeable batteries, and the docking station charges the rechargeable batteries. The portable compress device is operable to shut off automatically when connected to the docking station, when connected to a charger, when a pad assembly has been detached, or after a predetermined time interval has elapsed, for example.

According to one example, the portable compress device may include three or more surfaces and three or more pad attachment and detachment means, each corresponding to the three or more surfaces, arranged for application on a wide, or greater, treatment area.

According to a further example, the portable compress device may include two surfaces and two pad attachment and detachment means corresponding to the two surfaces. The surfaces may be arranged on opposing sides of the main body. The first surface is for hot therapy and the second surface is for cold therapy. A pressure sensor may detect which side is being used. In operation, current from the energy source may be directed to the surface being used.

In one example, the pad attachment and detachment means may include an inverted dome and a plurality of adjustable prongs for treating a treatment area comprising protruding skin such as a breast. The pad assembly may correspondingly include a plurality of openings to accommodate the prongs.

According to disclosed examples, the portable compress device may be used for the treatment of arthritis, blepharitis, breast-feeding, C-section inflammation and scar, fever, hot flashes, insect bites and stings, menstrual cramps, migraines, headaches, repetitive strain injury, or sports injuries comprising sprains and strains.

According to one example, the portable compress device may be used to treat a breast-feeding mother's skin to reduce symptoms such as an engorged tender breast, reduced milk flow, an obstructed milk duct, or discomfort from mastitis.

The present specification provides a portable compress device including a power source, a thermoelectric means for generating a temperature gradient on a surface, a controller means coupled with the thermoelectric means and the power source, a thermal sensor device configured to be in proximity to a user's skin at a treatment area, and a pad attachment and detachment means for attaching to and detaching a pad from the surface.

The device may be a handheld device. The device may include a refillable liquid dispenser secured to the said surface. The power source may include at least one battery, which may be rechargeable. The thermoelectric means, the thermal sensor device, the surface, and the pad may be configured together and operably coupled with other components of the device, enabling remote operation. The pad attachment and detachment means may include a mechanical configuration to interface with suitable structure formed on the pad.

According to one example, the pad includes a material that is essentially thermally conductive. The pad may be configured to interface with the device for attachment and removal to/from the surface. The pad may be impregnated with a salve. The pad may adhere to the skin after detachment from the device. The dispenser may transfer fluid from the dispenser to the pad secured on the surface.

In one example, the thermoelectric means is a Peltier effect device. The controller means may be located on an exterior surface of the device is operated by a user. The controller means may control thermal gradient polarity to provide warm and cool treatment instances. The controller means may control thermal gradient cycles to affect the duration of treatment instances. The thermal gradient profile modes may be in a range of 7° C.-60° C. The controller means may provide detection of battery level. Upon detecting a battery condition, the controller means may prohibit operation of the thermoelectric means. The controller means may include wireless communication to a portable electronic device running application code to interface with the portable compress device.

In one example, the device may include a retention strap that is configured along a length of the device enabling a user to obtain compression of the device on parts of a body of the user during use.

In some examples, the controller means may provide indication when a selected temperature is reached, end of a treatment instances, and one or more alarms.

The descriptions and drawings are not intended to limit the invention in any way and are provided as examples that may be substituted with similar means by one skilled in the art. The full scope and definition are contained in the following claims.

1. A portable compress device comprising: a main body, a power source; at least one electric means for generating a temperature gradient on a plurality of surfaces; a controller means coupled with the electric means and the power source; a thermal sensor device configured to be in proximity to a user's skin at a treatment area; and a plurality of pad attachment and detachment means for attaching and detaching a pad assembly to and from the plurality of surfaces, wherein the pad assembly is for moistened or dry application on the treatment area.

2. The portable compress device of claim 1 further comprising a plurality of heat sinks and a plurality of fans operable to control a temperature of the main body, wherein the main body is hand-held.

3. The portable compress device of claim 2 wherein the main body comprises a rubberized handle, and the main body and the pad assembly are curved to facilitate ergonomic operation.

4. The portable compress device of claim 1 further comprising a vibration motor.

5. The portable compress device of claim 1 wherein the pad assembly is selected from one of: a permanent gel pack that is integral with the portable compress device, and a disposable gel pack.

6. The portable compress device of claim 1 wherein the power source is selected from one of: disposable batteries, rechargeable batteries, AC power, solar power, and USB power.

7. A compress system comprising the portable compress device of claim 1 wherein the power source comprises rechargeable batteries, and the system comprises a docking station to charge the rechargeable batteries.

8. The compress system of claim 7 wherein the portable compress device is operable to shut off automatically in a condition selected from one of: when connected to the docking station, when connected to a charger, when a pad assembly has been detached, and after a predetermined time interval has elapsed.

9. The portable compress device of claim 1 comprising three or more surfaces and three or more pad attachment and detachment means, corresponding to the three or more surfaces, arranged for application on a wide treatment area.

10. The portable compress device of claim 1 comprising a first surface and a second surface and a first pad attachment and detachment means corresponding to first surface and a second pad attachment and detachment means corresponding to the second surface, arranged on opposing sides of the main body, wherein the first surface is for hot therapy and the second surface is for cold therapy, and further comprising a pressure sensor to detect which side is being used, wherein, in operation, a current is directed to the surface being used.

11. The portable compress device of claim 1 wherein the pad attachment and detachment means comprises an inverted dome and a plurality of adjustable prongs for treating the treatment area comprising protruding skin.

12. The portable compress device of claim 11 wherein the pad assembly comprises a plurality of openings to accommodate the adjustable prongs.

13. The portable compress device of claim 12 wherein protruding skin comprises a human breast or an animal breast.

14. A method of using a portable compress device to treat a condition selected from one of:
arthritis, blepharitis, breast-feeding, C-section inflammation and scar, fever, hot flashes, insect bites and stings, menstrual cramps, migraines, headaches, repetitive strain injury, and sports injuries comprising sprains and strains.

15. The method of using the portable compress device according to claim 14 to treat a breast-feeding mother's skin to reduce a symptom selected from one of: an engorged tender breast, reduced milk flow, an obstructed milk duct, and discomfort from mastitis.

16. A portable compress device comprising: a power source; a thermoelectric means for generating a temperature gradient on a surface; a controller means coupled with the thermoelectric means and the power source; a thermal sensor device configured to be in proximity to a user's skin at a treatment area; and a pad attachment and detachment means for attaching to and detaching a pad from the surface.

17. The device of claim 16, wherein the device is a handheld device comprising a refillable liquid dispenser secured to the said surface.

18. The device of claim 16, wherein the power source includes at least one battery.

19. The device of claim 18, wherein the battery is rechargeable.

20. The device of claim 16, wherein the thermoelectric means, the thermal sensor device, the surface, and the pad are configured together and operably coupled with other components of the device, enabling remote operation.

21. The device of claim 16, wherein the pad attachment and detachment means comprise a mechanical configuration to interface with suitable structure formed on the pad.

22. The device of claim 21, wherein the pad comprises a material that is essentially thermally conductive; and wherein the pad is configured to interface with the device for attachment and removal to/from the surface.

23. The device of claim 22, wherein the pad is impregnated with a salve.

24. The device of claim 22, wherein the pad adheres to the skin after detachment from the device.

25. The device of claim 17, wherein the dispenser transfers fluid from the dispenser to the pad secured on the surface.

26. The device of claim 16, wherein the thermoelectric means is a Peltier effect device.

27. The device of claim 16, wherein the controller means located on an exterior surface of the device is operated by a user.

28. The device of claim 17, wherein the controller means includes control of thermal gradient polarity to provide warm and cool treatment instances.

29. The device of claim 28, wherein the controller means includes control of thermal gradient cycles to affect the duration of treatment instances.

30. The device of claim 29, wherein the controller means includes plural thermal gradient profile modes in a range of 7° C.-60° C. and an off mode.

31. The device of claim 18, wherein the controller means provides detection of battery level.

32. The device of claim 31, wherein the controller means, upon detecting a battery condition, prohibits operation of the thermoelectric means.

33. The device of claim 16, wherein the controller means includes wireless communication to a portable electronic device running application code to interface with the portable compress device.

34. The device of claim 16, wherein the device further comprises a retention strap that is configured along a length of the device enabling a user to obtain compression of the device on parts of a body of the user during use.

35. The device of claim 16, wherein the controller means provides indication when a selected temperature is reached, end of a treatment instances, and one or more alarms.
